(19) United States
(12) Patent Application Publication
(10) Pub. No.: US 2022/0395184 A1
(43) Pub. Date: Dec. 15, 2022

(54) Heartrate Detection Device, Heartrate Detection Method, and Heartrate Detection Program

(71) Applicant: Nippon Telegraph and Telephone Corporation, Tokyo (JP)

(72) Inventors: Yuki Hashimoto, Tokyo (JP); Kei Kuwabara, Tokyo (JP); Nobuaki Matsuura, Tokyo (JP)

(21) Appl. No.: 17/775,121

(22) PCT Filed: Nov. 22, 2019
(86) PCT No.: PCT/JP2019/045793

§ 371 Date: May 6, 2022

(51) Int. Cl. A61B 5/0245; A61B 5/366

(52) U.S. Cl. CPC A61B 5/0245; A61B 5/366

(57) ABSTRACT

A heart rate detection device includes a sensor data acquisition device that acquires sensor data indicating an electrocardiogram of a living body to output a sequence of sampling data based on the sensor data, a first calculator that calculates, every sampling time, a time difference value of sampling data, a decision device that decides a heart rate time based on a change in the time difference value calculated by the first calculator exceeds a threshold that is set, a corrector that corrects the heart rate time based on when a cardiac potential becomes zero on a straight line passing through two points of sampling data at the heart rate time decided by the decision device and sampling data immediately before the sampling data at the heart rate time, and a second calculator that calculates a heart rate of the living body from the heart rate time that is corrected.

### CROSS-REFERENCE TO RELATED APPLICATIONS

This application is a national phase entry of PCT Application No. PCT/JP2019/045793, filed on Nov. 22, 2019, which application is hereby incorporated herein by reference.

### TECHNICAL FIELD

The present disclosure relates to a heart rate detection device, a heart rate detection method, and a heart rate detection program.

### BACKGROUND

In recent years, in various active scenes in daily lives such as running, and walking, there has been an increasing interest in wearing wearable devices to make his or her own health management by measurement data such as steps, activity, heart rate, and the like.

For example, PTL 1 discloses an application that acquires an electrocardiogram in wearing by a wearable sensor mounted on clothing, calculates a heart rate based on the acquired electrocardiogram, and transmits the obtained data to an external terminal such as a smartphone.

In such a related-art wearable device, because an external power supply is not used, power saving of the device is important in applications requiring extended continuous use, such as a case where the heart rate of a user is measured.

In order to achieve the power saving of the wearable device in the related art, an interval at which data such as the heart rate of the user is acquired is increased, and an amount of arithmetic processing per unit time is reduced.

For example, PTL 2 discloses a technique in which, in order to calculate the heart rate from a cardiac potential, a threshold is set according to the amplitude of an R wave for time series data of the cardiac potential, the R wave is detected when the data exceeds the threshold, and the heart rate is calculated from the cycle (R-R interval) in which the data exceeds the threshold.

For a method for detecting the heart rate of a stable living body without being affected by a body motion noise, a technique has also been proposed in which, instead of using time series data of the cardiac potential, a time difference value of the cardiac potential and a time difference value taking into account clearance before and after a peak of a QRS wave are used as index values (see PTL 3).

### CITATION LIST

### Patent Literature

PTL 1: WO 2016/024495
PTL 2: JP 2015-156936 A
PTL 3: JP 6404784 B.

### SUMMARY

### Technical Problem

However, in the related-art techniques, because a characteristic amount of the cardiac potential is detected by the threshold, a shift in detection time corresponding to the acquisition interval of the data is generated, which may lead to errors in calculating the heart rate.

The present disclosure has been made to solve the above-described problem, and an object of the present disclosure is to more accurately measure the heart rate of the living body.

### Means for Solving the Problem

In order to solve the above-described problem, a heart rate detection device according to the present disclosure includes a sensor data acquisition unit that acquires sensor data indicating an electrocardiogram of a living body to output a sequence of sampling data based on the sensor data, a first calculation unit that calculates, every sampling time, a time difference value of sampling data from the sequence of sampling data that is output, a decision unit that decides a heart rate time based on time at which a change in the time difference value calculated by the first calculation unit exceeds a threshold that is set, a correction unit that corrects the heart rate time with time at which a cardiac potential becomes zero on a straight line passing through two points of sampling data at the heart rate time decided by the decision unit and sampling data immediately before the sampling data at the heart rate time as reference time, and a second calculation unit that calculates a heart rate of the living body from the heart rate time corrected by the correction unit.

In order to solve the above-described problem, a heart rate detection method according to the present disclosure includes acquiring sensor data indicating an electrocardiogram of a living body to output a sequence of sampling data based on the sensor data, calculating, every sampling time, a time difference value of sampling data from the sequence of sampling data that is output in the acquiring, deciding a heart rate time based on time at which a change in the time difference value calculated in the calculating the time difference value exceeds a threshold that is set, correcting the heart rate time with time at which a cardiac potential becomes zero on a straight line passing through two points of sampling data at the heart rate time decided in the deciding and sampling data immediately before the sampling data at the heart rate time as reference time, and calculating a heart rate of the living body from the heart rate time corrected in the correcting.

In order to solve the above-described problem, a heart rate detection program according to the present disclosure causing a computer to execute acquiring sensor data indicating an electrocardiogram of a living body to output a sequence of sampling data based on the sensor data, calculating, every sampling time, a time difference value of sampling data from the sequence of sampling data that is output in the acquiring, deciding a heart rate time based on time at which a change in the time difference value calculated in the calculating the time difference value exceeds a threshold that is set, correcting the heart rate time with time at which a cardiac potential becomes zero on a straight line passing through two points of sampling data at the heart rate time decided in the deciding and sampling data immediately before the sampling data at the heart rate time as reference time, and calculating a heart rate of the living body from the heart rate time corrected in the correcting.

### Effects of Embodiments of the Invention

According to the present disclosure, a heart rate time is corrected with time at which a cardiac potential becomes zero on a straight line passing through two points of sampling data at the heart rate time decided based on time exceeding a set threshold at which a change in a time difference value of sampling data in each sampling time calculated from a sequence of sampling data based on an electrocardiogram and sampling data immediately before the sampling data at the heart rate time as reference time. Therefore, the number of heart beats of the user can be more accurately measured.

### BRIEF DESCRIPTION OF THE DRAWINGS

FIG. 1 is a block diagram illustrating a functional configuration of a heart rate detection device according to an embodiment of the present disclosure.

FIG. 2 is a diagram explaining an electrocardiogram according to the embodiment.

FIG. 3 is a diagram explaining the electrocardiogram according to the embodiment.

FIG. 4 is a block diagram illustrating a functional configuration of a heart rate time calculation unit according to the embodiment.

FIG. 5 is a block diagram illustrating another example of the functional configuration of the heart rate time calculation unit according to the embodiment.

FIG. 6 is a block diagram illustrating an example of a hardware configuration of the heart rate detection device according to the embodiment.

FIG. 7 is a flowchart explaining a heart rate detection method according to the embodiment.

FIG. 8 is a flowchart explaining heart rate time calculation processing according to the embodiment.

FIG. 9 is a diagram explaining an effect of the heart rate detection device according to the embodiment.

### DETAILED DESCRIPTION OF ILLUSTRATIVE EMBODIMENTS

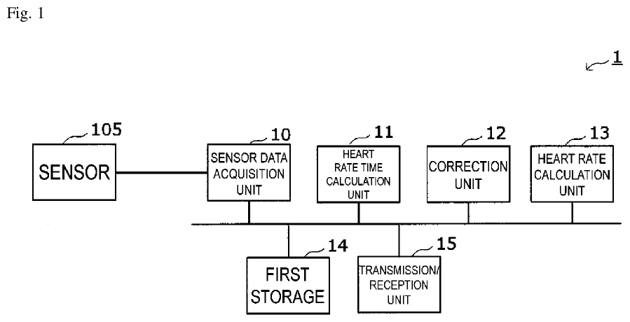

Hereinafter, a preferred embodiment of the present disclosure will be described in detail with reference to FIGS. 1 to 9.

### Summary of Embodiments of the Invention

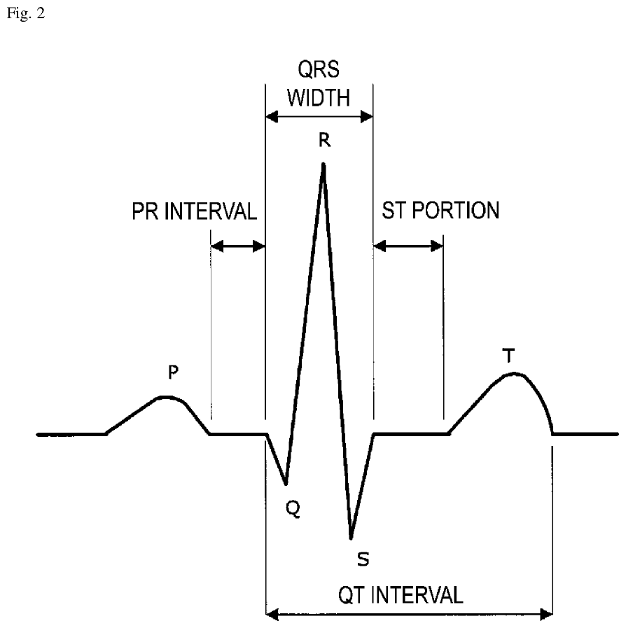

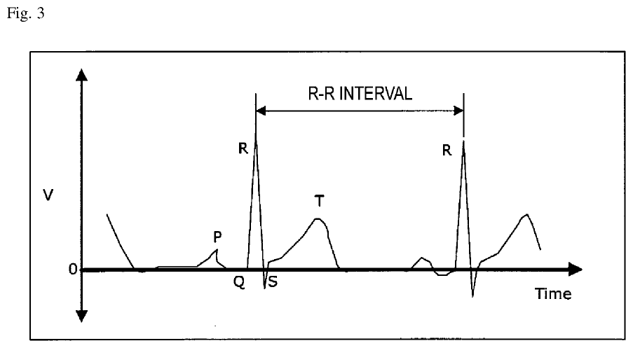

An outline of a heart rate detection device 1 according to an embodiment of the present disclosure will be described with reference to FIGS. 2 and 3.

FIGS. 2 and 3 are diagrams illustrating a typical electrocardiogram (ECG). An ECG waveform is composed of a continuous heart rate waveform, and one heart rate waveform is composed of components such as a P wave, a Q wave, an R wave, an S wave, and a T wave, which reflect an activity of each atrium and each ventricle, as illustrated in FIG. 2. Of these, the R wave is accompanied with ventricular contraction (depolarization of a ventricular muscle) and has a large amplitude, so that detection of the heart rate is often performed with the R wave as a guide. In particular, a steep change from the R wave to the S wave is emphasized in a peak shape by taking a time differential of a sequence of sampling data of the ECG waveform, which allows the heart rate to be easily detected. An interval between heart rates per one beat is referred to as an R-R interval and is treated as a primary index of a heart rate variability.

In the heart rate detection device 1 of the embodiment, a detection point is set onto an RS interval from the beginning of the QRS wave included in the ECG waveform to a peak of the S wave in order to detect the steep change from the R wave to the S wave. In the embodiment, the time at which the electric potential becomes zero during the RS interval is estimated using the detection point on the RS interval and the cardiac potential corresponding to the point immediately before the detection point, and the R-R interval is corrected using the time interval of the estimated time.

A configuration of the heart rate detection device 1 of the embodiment will be described below with reference to FIG. 1. A procedure in which the heart rate detection device 1 detects one heart rate to obtain a heart rate time of the heart rate will be described below. Time series data of the heart rate time is obtained by repeating the calculation of the heart rate time over a duration of the ECG waveform, and the R-R interval can be calculated from the time series data.

In the embodiment, sampling data obtained by sampling the ECG waveform is set to D(i). At this point, i (i=1, 2, . . . ) indicates a number given in sequence to the data for one sampling. The larger the number i of the data row, the later the sampling time.

In the embodiment, it is assumed that a minimum value of a time difference value in the range of a first time interval Tinr1 is Min1, that a minimum value of a time difference value in the range of a second time interval Tinr2 is Min2, and that a minimum value of a time difference value in the range of a third time interval Tinr3 is Min3, described below. For example, the initial values of these minimum values Min1, Min2, and Min3 are set to 0.

### Functional Block of Heart Rate Detection Device

As illustrated in FIG. 1, the heart rate detection device 1 includes a sensor data acquisition unit 10, a heart rate time calculation unit 11, a correction unit 12, a heart rate calculation unit (second calculation unit) 13, a first storage 14, and a transmission/reception unit 15.

The sensor data acquisition unit 10 acquires sensor data including an electrocardiographic signal and a measurement time of a user. For example, the sensor data acquisition unit 10 may acquire the electrocardiographic signal of the user measured by a sensor 105 composed of an electrocardiograph. The sensor data acquisition unit 10 performs signal processing, such as amplification, AD conversion, and noise removal of the acquired electrocardiographic signal. The sensor data acquisition unit 10 stores the sampling data D(i) that is the time series data of the ECG and the sampling time in the first storage 14. The case where the sensor data acquisition unit 10 acquires the electrocardiographic signal of the user from the sensor 105 is described in the embodiment. However, the sensor data acquisition unit 10 may acquire the previously measured electrocardiographic signal of the user in an off-line manner.

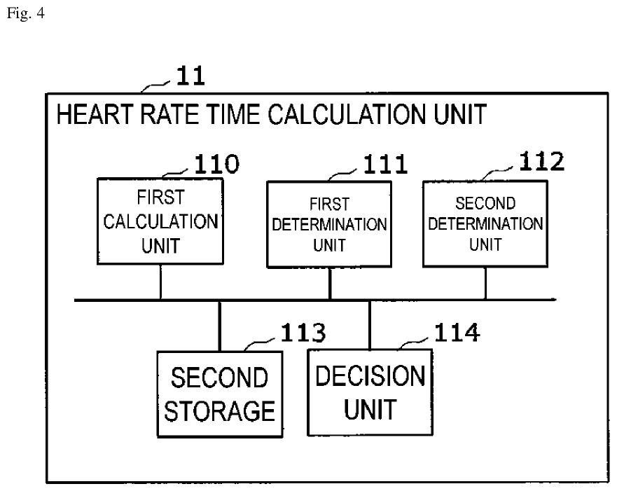

The heart rate time calculation unit 11 reads the sampling data D(i) of the ECG waveform stored in the first storage 14 and calculates the heart rate time. As illustrated in FIG. 4, the heart rate time calculation unit 11 includes a first calculation unit 110, a first determination unit 111, a second determination unit 112, a second storage 113, and a decision unit 114 (see PTL 3).

The first calculation unit 110 calculates a time difference value DY(i) of the sampling data D(i) of the ECG waveform for each sampling time (time indicated by i). More specifically, the first calculation unit 110 reads data D(i+1) that is one sampling after the sampling data D(i) of the ECG waveform and the sampling data D(i−1) that is one sampling before the sampling data D(i) of the ECG waveform from the first storage 14, and calculates the time difference value DY(i) of the sampling data D(i) from the following Equation (1).

DY(i)=D(i+1)−D(i−1) (1)

The first determination unit 111 determines whether the time difference value DY(i) calculated by the first calculation unit 110 is below a threshold TH. In the embodiment, a peak of the time difference value DY(i) due to the steep change from the R wave to the S wave is detected as illustrated in the ECG waveforms of FIGS. 2 and 3. The peak of the time difference value DY(i) appears as a negative value. Accordingly, the first determination unit 111 performs threshold processing on the time difference value DY(i) using the threshold TH of the set negative value.

For example, the first determination unit 111 can use, as the threshold TH, a value obtained by multiplying the last peak (Min2), e.g., the average value of the last five peaks (Min2), of the time difference value DY(i) detected as the heart rate by 0.5. The reason for multiplying the average value of the peak (Min2) by 0.5 is not to simply detect the peak, but to set the absolute value of the threshold TH to a smaller value (see NPL 3). A peak having a small absolute value can also be captured by reducing the absolute value of the threshold TH.

The second determination unit 112 determines whether a first elapsed time T1 from the heart rate time immediately before to the latest sampling time (the time indicated by i) at which the time difference value DY(i) is obtained is within the range of the first time interval Tinr1. The second determination unit 112 determines whether a second elapsed time T2 from the time at which the time difference value DY(i) falls below the threshold TH to the latest sampling time at which the time difference value DY(i) is obtained is within the range of the second time interval Tinr2. Furthermore, the second determination unit 112 determines whether a third elapsed time T3 from the time at which the second elapsed time T2 exceeds the range of the second time interval Tinr2 to the latest sampling time at which the time difference value DY(i) is obtained is within the range of the third time interval Tinr3.

The first time interval Tinr1 is a parameter used to define a time domain prior to the next assumed heart rate time. The second time interval Tinr2 is a parameter used to define a time domain assumed to include the peak of the time difference value DY(i). The third time interval Tinr3 is a parameter used to define a certain time domain after the time domain assumed to include the peak of the time difference value DY(i).

For example, the second determination unit 112 uses the time from the time that is shorter than the R-R interval obtained from the heart rate time immediately before by 150 [ms] to the time at which 100 [ms] was added to the time that is shorter than the R-R interval obtained from the heart rate time immediately before by 150 [ms] as the range of the first time interval Tinr1. At this point, the R-R interval is time obtained by subtracting the previous heart rate time of the heart rate time immediately before from the heart rate time immediately before.

Alternatively, the second determination unit 112 can use the time domain from the heart rate time immediately before until the time immediately before the time difference value DY(i) next exceeds the threshold TH as the range of the first time interval Tinr1.

The second time interval Tinr2 is preferably a sufficient time width covering the peak of the time difference value DY(i), and for example, can be previously set to 50 [ms].

For example, the third time interval Tinr3 can be previously set to 100 [ms].

The second storage 113 holds the minimum value Min1 of the time difference value DY(i) when the first elapsed time T1 is within the range of the first time interval Tinr1. The second storage 113 holds the minimum value Min2 of the time difference value DY(i) when the second elapsed time T2 is within the range of the second time interval Tinr2. The second storage 113 holds the minimum value Min3 of the time difference value DY(i) when the third elapsed time T3 is within the range of the third time interval Tinr3.

When the relationship between the minimum value Min1, Min2, and Min3 of the time difference value DY(i) held in the second storage 113 satisfies the previously-set condition in which the heart rate time is established, the decision unit 114 sets the time at which the time difference value DY(i) falls below the threshold TH, or the time at which the minimum value Min2 is obtained to the heart rate time. Whether the decision unit 114 uses any of the times as the heart rate time can be previously set by a program.

For example, the fact that both a ratio Min2/Min1 of the minimum value Min2 to the minimum value Min1 and a ratio Min2/Min3 of the minimum value Min2 to the minimum value Min3 exceed a certain value can be used as the previously-set condition for establishing the heart rate time. Whether the peak of the time difference value is unimodal can be determined by setting previously such a condition.

The correction unit 12 specifies time at which the cardiac potential becomes zero using the ECG waveform data based on the heart rate time calculated by the heart rate time calculation unit 11. The correction unit 12 uses the specified time at which the cardiac potential becomes zero as the reference time used to calculate the R-R interval. More specifically, the correction unit 12 specifies time at which the cardiac potential becomes zero on a straight line passing through two points of an ECG waveform data D(n) at the heart rate time calculated by the heart rate time calculation unit 11 and an ECG waveform data D(n−1) immediately before the ECG waveform data D(n), and set the time to the reference time used to calculate the R-R interval.

For example, when the heart rate time of the time at which the cardiac potential becomes zero is used as a reference, a deviation Δn (in units of sampling data number) is calculated from the following Equation (2).

$$\begin{matrix}Math 1 & \\ Δ n = - \frac{D ( n ) - D ( n - 1 )}{D ( n )} & ( 2 )\end{matrix}$$

The correction unit 12 can calculate the reference time using the deviation Δn calculated by the Equation (2) above.

At this point, each of the ECG waveform data D(n) at the heart rate time calculated by the heart rate time calculation unit 11 and the ECG waveform data D(n−1) immediately before the ECG waveform data D(n) needs to be a point on the RS interval. That is, there is an upper limit for the sampling interval. For example, the QRS interval is typically said to be approximately 100 [ms], and in consideration of this, the RS interval is approximately 25 [ms], so that the sampling interval needs to be less than or equal to 12.5 [ms].

On the other hand, when the sampling interval is too small, the ECG waveform data D(n) at the heart rate time calculated by the heart rate time calculation unit 11 and the ECG waveform data D(n−1) immediately before the ECG waveform data D(n) are present in the region where the slope of the tangent near the S wave changes steeply. Thus, an error increases when the heart rate time is corrected using two points passing through the ECG waveform data D(n) and the ECG waveform data D(n−1) immediately before the ECG waveform data D(n). That is, there is also a lower limit for the sampling interval, and specifically the lower limit is approximately 4 [ms].

The heart rate calculation unit 13 calculates a heart rate X [bpm] from the latest heart rate time corrected by the correction unit 12. Specifically, the heart rate calculation unit 13 calculates the instantaneous heart rate X by the following Equation (3), when the R-R interval that is the time subtracting the heart rate time immediately before the latest heart rate time calculated and established by the correction unit 12 from the latest heart rate time is set to RRI [ms].

$$\begin{matrix}Math 2 & \\ X = \frac{60000}{R R 1} & ( 3 )\end{matrix}$$

Instead of the instantaneous heart rate, the heart rate calculation unit 13 can be configured to calculate the average heart rate X using the following Equation (4) that is described in Reference Literature 3 (JP 2018-011819 A).

Math 3

X(i)=q×HR(i)+(1−q)×X(i−1) (4)

In the Equation (4) above, HR(i) represents the i-th instantaneous heart rate before averaging processing, X(i−1) represents the average heart rate averaging up to (i−1)-th instantaneous heart rate, 1 represents a predetermined average coefficient, and X(i) represents the average heart rate averaging up to the i-th instantaneous heart rate.

The transmission/reception unit 15 transmits the heart rate calculated by the heart rate calculation unit 13 to an external terminal device (not illustrated) such as an external smart phone by wire or wireless.

### Another Example of Heart Rate Time Calculation Unit

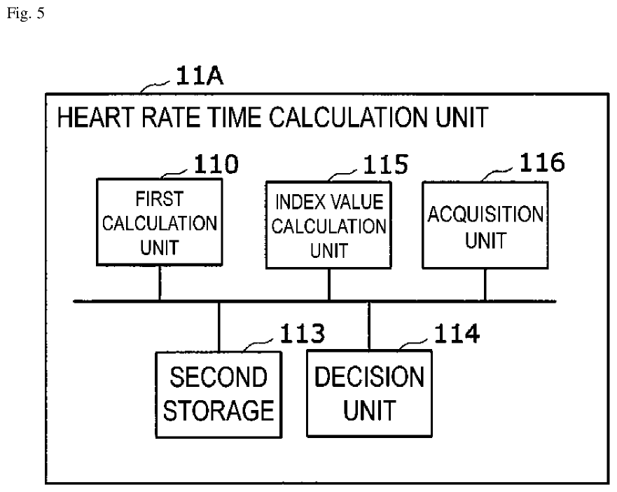

The heart rate detection device 1 of the embodiment is not limited to the functional configuration provided by the heart rate time calculation unit 11 described above. For example, the heart rate time can be calculated by a heart rate time calculation unit 11A having a configuration illustrated in FIG. 5 (see Reference Literature 1: JP 6360017 B and Reference Literature 2: JP 6527286 B).

The heart rate time calculation unit 11A includes the first calculation unit 110, an index value calculation unit 115, an acquisition unit 116, the second storage 113, and the decision unit 114.

The first calculation unit 110 calculates the time difference value DY(i) of the sampling data D(i) of the electrocardiogram for each sampling time.

The acquisition unit 116 acquires, for each sampling point i, the minimum value of the time difference value in the predetermined time domain before and after the sampling point i.

The index value calculation unit 115 obtains, for each sampling point i, the value obtained by subtracting the minimum value of the time difference value in the predetermined time domain before and after the sampling point i from the time difference value DY(i) at the sampling point i as an index value.

The decision unit 114 specifies the index value of a point that is below a predetermined threshold and at which a trend in change of the index value changes from the decrease to the increase as a downward peak among the index values for each sampling point i, and sets the time of the specified downward peak to the heart rate time.

For example, the predetermined time domain before and after the sampling point i is a region of −112.5 ms to −12.5 ms and a region of +12.5 ms to +112.5 ms with respect to the time of the sampling point i.

The second storage 113 temporarily stores the minimum value of the time difference value acquired by the acquisition unit 116, the index value obtained by the index value calculation unit 115, and the heart rate time decided by the decision unit 114.

### Hardware Configuration of Heart Rate Detection Device

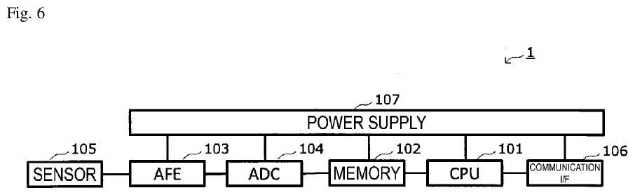

An example of a hardware configuration of the heart rate detection device 1 having the above-described functions will be described below with reference to FIG. 6.

As illustrated in FIG. 6, for example, the heart rate detection device 1 can be implemented by a computer including a CPU 101, a memory 102, an AFE 103, an ADC 104, and a communication I/F 1o6 and a program controlling these hardware resources. In the heart rate detection device 1, for example, the sensor 105 provided outside is connected through a bus. The heart rate detection device 1 includes a power supply 107, and the power supply 107 supplies power to the entire device other than the sensor 105 in FIG. 5.

A program causing the CPU 101 to perform various controls or calculations is previously stored in the memory 102. Each function of the heart rate detection device 1 including the sensor data acquisition unit 10, the heart rate time calculation unit 11, the correction unit 12, and the heart rate calculation unit 13 in FIG. 1 is implemented by the CPU 101 and the memory 102.

The sensor 105 is implemented with the electrocardiograph or the like, and measures a faint electrocardiographic signal through a skin of the user.

The analog front end (AFE) 103 is a circuit that amplifies the electrocardiographic signal that is the analog signal measured by the sensor 105.

The analog-to-digital converter (ADC) 104 is a circuit that converts an analog signal amplified by the AFE 103 into a digital signal at a predetermined sampling frequency. The AFE 103 and the ADC 104 implement the sensor data acquisition unit 10 in FIG. 1.

The memory 102 is implemented by a non-volatile memory such as a flash memory, a volatile memory such as a DRAM, and the like. The memory 102 temporarily stores time series data of signals output from the ADC 104. The memory 102 implements the first storage 14 in FIG. 1 and the second storage in FIG. 5.

The memory 102 includes a program storage region in which a program used by the heart rate detection device 1 to perform heart rate detection processing is stored. Further, for example, a backup area for backing up the data, programs, and the like described above may be provided.

The communication I/F 106 is an interface circuit performing communication with various external electronic devices through a communication network NW.

For example, a communication interface compatible with a wired or wireless data communication standard such as LTE, 3G, Bluetooth (trade name), Bluetooth Low Energy, and Ethernet (trade name) and an antenna are used as the communication I/F 106. The transmission/reception unit 15 in FIG. 1 is implemented by the communication I/F 106.

The heart rate detection device 1 acquires time information from a clock incorporated in the CPU 101 or a time server (not illustrated), and uses the time information as the sampling time.

### Heart Rate Detection Method

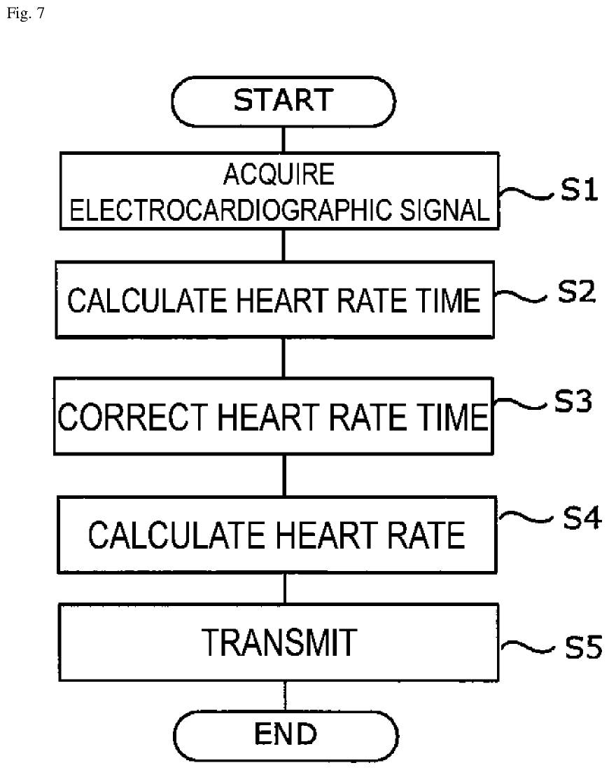

The operation of the heart rate detection device 1 having the above configuration will be described below with reference to a flowchart in FIG. 7. When the sensor 105 consisting of the electrocardiograph is attached to the user and the measurement of the electrocardiographic signal is started, the following processing is executed.

The sensor data acquisition unit 10 acquires the electrocardiographic signal of the user from the sensor 105 (step S1). The sensor data acquisition unit 10 amplifies the electrocardiographic signal, performs sampling at a predetermined sampling frequency, and outputs the digital ECG waveform. The sampling data of the ECG waveform is stored in the first storage 14.

Subsequently, the heart rate time calculation unit 11 reads the sampling data D(i) of the ECG waveform from the first storage 14 and calculates the heart rate time (step S2).

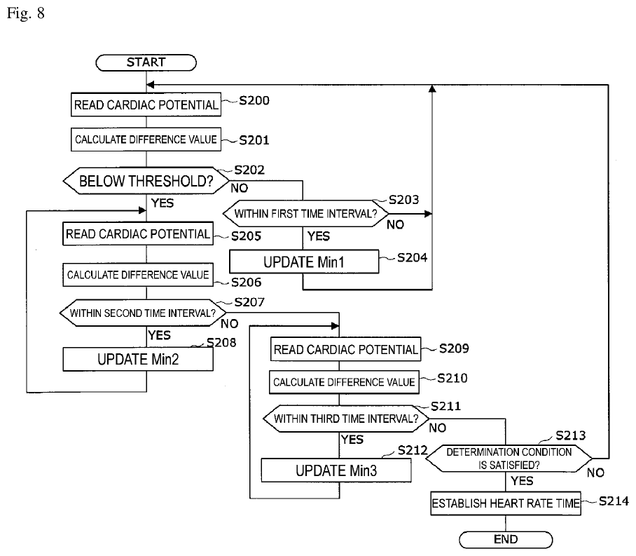

Heart rate time calculation processing executed by the heart rate time calculation unit 11 in step S2 will now be described with reference to FIG. 8.

In order to calculate the time difference value DY(i) of the sampling data D(i), the first calculation unit 110 reads the data D(i+1) that is one sampling after the sampling data D(i) and the data D(i−1) that is one sampling before the sampling data D(i) from the first storage 14 (step S200).

Subsequently, the first calculation unit 110 calculates the time difference value DY(i) of the sampling data D(i) using the Equation (1) (step S201). Subsequently, the first determination unit 111 determines whether the time difference value DY(i) is below the threshold TH (step S202).

Subsequently, when the time difference value DY(i) is determined to be not below the threshold TH (NO in step S202), the second determination unit 112 determines whether the first elapsed time T1 that is the elapsed time from the heart rate time immediately before to the sampling time (time indicated by i) of the sampling data D(i) to be processed is within the range of the first time interval Tinr1 (step S203).

When the first elapsed time T1 is determined to be within the range of the first time interval Tinr1, namely, when the sampling time of the sampling data D(i) to be processed is in the time domain prior to the assumed next heart rate time (YES in step S203), the second storage 113 updates the minimum value Min1 of the time difference value within the range of the first time interval Tinr1 (step S204). That is, the second storage 113 compares the time difference value DY(i) of the sampling data D(i) to be processed and the current minimum value Min1, and sets the time difference value DY(i) to the new minimum value Min1 when the time difference value DY(i) to be processed is smaller than the current minimum value Min1. Then, the processing returns to step S200. When the first elapsed time T1 is determined to be outside the range of the first time interval Tinr1 in step S203, the processing returns to step S200 without updating the minimum value Min1.

On the other hand, when the time difference value DY(i) is determined to exceed the threshold TH (YES in step S202), the first calculation unit 110 reads the data D(j+1) that is one sampling after the sampling data D(j) and the data D(j−1) that is one sampling before the sampling data D(j) from the first storage 14 in order to calculate the time difference value DY(j) of the next sampling data D(j) (step S205). Here, j is an integer of j a (i+1), and the initial value is j=i+1. Then, the first calculation unit 110 calculates the time difference value DY(j) of the sampling data D(j) in the same manner as the Equation (1) above (step S206).

The time determination unit 5 determines whether the elapsed time (second elapsed time T2) from the time at which the time difference value Y(i) exceeds the threshold TH to the sampling time (time indicated by j) of the sampling data X(j) to be processed is within the range of the second time interval Tinr2 (step S207).

When the second elapsed time T2 is determined to be within the range of the second time interval Tinr2, namely, when the sampling time of the sampling data D(j) to be processed is within the time domain assumed to include the peak of the time difference value (YES in step S207), the second storage 113 updates the minimum value Min2 of the time difference value within the range of the second time interval Tinr2 (step S208). That is, the second storage 113 compares the time difference value DY(j) of the sampling data D(j) to be processed and the current minimum value Min2, and sets the time difference value DY(j) to the new minimum value Min2 when the time difference value DY(j) to be processed is smaller than the current minimum value Min2. Then, the processing returns to step S205.

In this way, the pieces of processing in steps S205 to S2o8 are repeatedly executed while the sampling time transfers the processing target to the new sampling data D(j) one by one like j=i+1, i+2, i+3, i+4, . . . . until the second elapsed time T2 becomes outside the range of the second time interval Tinr2.

Subsequently, when it is determined in step S207 that the second elapsed time T2 is past the second time interval Tinr2, namely, when the sampling time exceeds the time domain assumed to include the peak of the time difference value, the first calculation unit 110 reads the data D(k+1) that is one sampling after the sampling data D(k) and the data D(k−1) that is one sampling before the sampling data D(k) from the first storage 14 in order to calculate the time difference value DY(k) of the next sampling data D(k) (step S209). Here, k is an integer of k≥(j+1), and the initial value is k=j+1). The first calculation unit 110 calculates the time difference value DY(k) of the sampling data D(k) in the same manner as in the Equation (1) above (step S210).

The second determination unit 112 determines whether the elapsed time (third elapsed time T3) from the time at which the second elapsed time T2 determined to exceed the range of the second time interval Tinr2 in step S207 to the sampling time (time indicated by k) of the sampling data D(k) to be processed is within the range of the third time interval Tinr3 (step S211).

When the third elapsed time T3 is determined to be within the range of the third time interval Tinr3, namely, when the sampling time of the sampling data D(k) to be processed is within the certain time domain after the peak of the time difference value (YES in step S211), the second storage 113 updates the minimum value Min3 of the time difference value within the range of the third time interval Tinr3 (step S212). That is, the second storage 113 compares the time difference value DY(k) of the sampling data D(k) to be processed and the current minimum value Min3, and sets the time difference value DY(k) to the new minimum value Min3 when the time difference value DY(k) to be processed is smaller than the current minimum value Min3. Then, the processing returns to step S209.

In this way, the pieces of processing in steps S209 to S212 are repeatedly executed while the sampling time transfers the processing target to the new sampling data D(k) one by one like k=j+1, j+2, j+3, j+4, . . . . until the third elapsed time T3 becomes outside the range of the third time interval Tinr3.

Subsequently, when the third elapsed time T3 is determined to be outside the range of the third time interval Tinr3 at step S211, namely, when the sampling time of the sampling data D(k) to be processed exceeds the certain time domain after the peak of the time difference value, the decision unit 114 determines whether the relationship of the three minimum values Min1, Min2, Min3 satisfies the previously-set condition that establishes the heart rate time (step S213).

Here, the minimum value Min2 corresponds to the magnitude of the detected peak in the time difference value of the sampling data, the minimum value Min1 corresponds to a floor level of a certain region before the peak, and the minimum value Min3 corresponds to a floor level of a certain region after the peak. When the peak of the time difference value is a unimodal negative peak, the minimum value Min2 needs to have the absolute value that is greater than the minimum values Min1 and Min3.

The decision unit 114 can determine that the predetermined condition is satisfied when both the ratio Min2/Min1 and the ratio Min2/Min3 exceed a certain value, and the decision unit 114 can determine that the predetermined condition is not satisfied when at least one of the ratio Min2/Min1 and the ratio Min2/Min3 is less than or equal to the certain value.

When the relationship of the three minimum values Min1, Min2, Min3 is determined to satisfy the condition for establishing the heart rate time (YES in step S213), the decision unit 114 adopts, as the heart rate time, the time at which the time difference value DY(i) exceeds the threshold TH or the latest time (the time of the peak of the time difference value) at which the minimum value Min2 is updated (step S214).

After completion of step S214, the processing returns to step S200 as i=k+1. Thus, the next heart rate detection is started. When it is determined in step S213 that the relationship of the three minimum values Min1, Min2, Min3 does not satisfy the condition for establishing the heart rate time, the processing also returns to step S200 as i=k+1. In this case, the time difference value exceeding the threshold TH detected in step S202 is due to not the heart rate but a noise, and the heart rate is not yet detected.

Thus, the time series data of the heart rate time is obtained by repeating the pieces of processing in steps S200 to S214.

Subsequently, returning to FIG. 7, the correction unit 12 corrects the heart rate time obtained by the heart rate time calculation unit 11 (step S3). More specifically, the correction unit 12 specifies the time at which the cardiac potential becomes zero from the ECG waveform data based on the heart rate time calculated by the heart rate time calculation unit 11. The correction unit 12 uses the specified time at which the cardiac potential becomes zero as the reference time used to calculate the R-R interval. More specifically, the correction unit 12 specifies time at which the cardiac potential becomes zero on a straight line passing through two points of an ECG waveform data D(n) at the heart rate time calculated by the heart rate time calculation unit 11 and an ECG waveform data D(n−1) immediately before the ECG waveform data D(n), and set the time to the reference time used to calculate the R-R interval.

Subsequently, the heart rate calculation 13 calculates the heart rate X [bpm] from the latest heart rate time corrected using the reference time at which the cardiac potential becomes zero specified by the correction unit 12 (step S4). More specifically, the heart rate calculation unit 13 calculates the instantaneous heart rate X using the Equation (3) described above, with the R-R interval that is the time obtained by subtracting the heart rate time immediately before the latest heart rate time calculated and established by the correction unit 12 from the latest heart rate time as RRI [ms]. Alternatively, the heart rate calculation unit 13 may be configured to calculate the average heart rate X using the Equation (4) above instead of the instantaneous heart rate.

Thereafter, the transmission/reception unit 15 transmits the instantaneous heart rate X or the average heart rate X calculated by the heart rate calculation unit 13 to an external terminal (not illustrated) such as a smartphone via a communication network (step S5).

### Effect of Heart Rate Detection Device

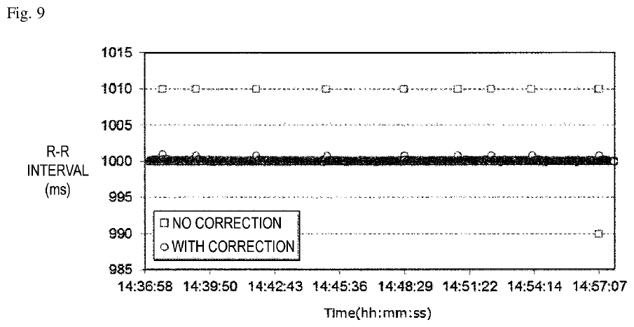

An effect of the heart rate detection device 1 of the embodiment will be described below with reference to FIG. 9. In the example of FIG. 9, a detection result of the R-R interval (RRI) is illustrated by the presence or absence of the correction unit 12 when a constant electrocardiogram is input with a heart rate of 60 [bpm]. In FIG. 9, a horizontal axis represents time and a vertical axis represents R-R interval. The “square” marker indicates the measurement results obtained by the heart rate detection device that is not provided with the correction unit 12 according to a related-art example. On the other hand, the “round” marker indicates the measurement result obtained by the heart rate detection device 1 that is provided with the correction unit 12 according to the embodiment.

In the example of FIG. 9, the heart rate time calculated by the heart rate time calculation unit 11A described in Reference Literature 1 and Reference Literature 2 as the heart rate time calculation unit 11 was used. The sampling interval was 10 [ms]. It can be seen from the calculation results of FIG. 9 that a detection error is reduced by introducing the correction unit 12. In the heart rate detection device 1 of the embodiment, it is demonstrated that the heart rate and the RRI can be detected from the ECG waveform with accuracy exceeding the sampling interval.

As described above, according to the heart rate detection device 1 of the embodiment, the time at which the electrical potential becomes zero at the RS interval is estimated using the cardiac potential corresponding to the detection point and the point immediately before the detection point on the RS, and the R-R interval is corrected using the time interval of the estimated time, so the heart rate of a living body can be measured more accurately.

In addition, according to the embodiment, the heart rate of the user can be accurately detected even when the acquisition interval of data is set longer in obtaining the heart rate of the user using the electrocardiograph in order to save power.

In the described embodiment, the case where the heart rate is detected has been illustrated. However, the present disclosure can be applied not only for the heart rate, but also for the calculation of the heart rate based on periodic biological information such as a pulse wave or for the correction of other characteristic amounts of the R-R interval included in the ECG waveform, for example, the sample data of the P waves, the Q waves, the S waves, and the T waves.

Additionally, in the embodiment, the case in which the technique described in PTL 3 and Reference Literatures 1, 2 is used as the specific example of the heart rate time calculation unit 11. However, the heart rate detection device 1 of the embodiment can apply the heart rate time calculation unit 11 that is not limited to the method for calculating the heart rate time described in these Literatures.

In addition, the method for calculating the heart rate time described in PTL 3 and Reference Literatures 1, 2 can reduce the false heart rate detection due to fluctuation of a baseline and the steep noise caused by body movement, perspiration, and the like as compared to the related-art heart rate detection technique. For this reason, the heart rate can be calculated with higher accuracy by combining the heart rate time calculation method described in PTL 3 and Reference Literatures 1, 2 and the correction unit 12 of the present disclosure.

Although the embodiment of the heart rate detection device, the heart rate detection method, and the heart rate detection program of the present disclosure have been described above, the present disclosure is not limited to the described embodiment, and various modifications that can be assumed by those skilled in the art can be made in the scope of the disclosure described in the aspects.

### REFERENCE SIGNS LIST

1 Heart rate detection device
10 Sensor data acquisition unit
11,11A Heart rate time calculation unit
12 Correction unit
13 Heart rate calculation unit
14 First storage
15 Transmission/reception unit
110 First calculation unit
111 First determination unit
112 Second determination unit
113 Second storage
114 Decision unit
115 Index value calculation unit
116 Acquisition unit
101 CPU
102 Memory
103 AFE
104 ADC
105 Sensor
106 Communication I/F
107 Power supply.

1.-7. (canceled)

8. A heart rate detection device comprising:
a sensor data acquisition device configured to acquire sensor data indicating an electrocardiogram of a living body to output a sequence of sampling data based on the sensor data;
a first calculator configured to calculate, every sampling time, a time difference value of sampling data from the sequence of sampling data that is output;
a decision device configured to decide a heart rate time based a change in the time difference value calculated by the first calculator exceeds a threshold that is set;
a corrector configured to correct the heart rate time with time at which a cardiac potential becomes zero on a straight line passing through two points of sampling data at the heart rate time decided by the decision device and sampling data immediately before the sampling data at the heart rate time as reference time; and
a second calculator configured to calculate a heart rate of the living body from the heart rate time corrected by the corrector.

9. The heart rate detection device according to claim 8, wherein the two points of the sampling data at the heart rate time decided by the decision device and the sampling data immediately before the sampling data at the heart rate time are points on an RS interval from a beginning of a QRS wave composed of a Q wave, an R wave, and an S wave included in the electrocardiogram to a peak of the S wave, and
the corrector corrects the heart rate time with time at which the cardiac potential becomes zero on the RS interval as the reference time.

10. The heart rate detection device according to claim 9, wherein the second calculator calculates an R-R interval that is a time interval between an R wave and a previous R wave of the R wave included in the electrocardiogram of the living body using the reference time obtained by the corrector to calculate the heart rate of the living body from the R-R interval.

11. The heart rate detection device of claim 8, wherein the heart rate calculated by the second calculator is an instantaneous heart rate.

12. The heart rate detection device of claim 8, wherein the heart rate calculated by the second calculator is an average heart rate.

13. A heart rate detection method comprising:
acquiring sensor data indicating an electrocardiogram of a living body to output a sequence of sampling data based on the sensor data;
calculating, every sampling time, a time difference value of sampling data from the sequence of sampling data that is output in the acquiring;
deciding a heart rate time based on time at which a change in the time difference value calculated in the calculating the time difference value exceeds a threshold that is set;
correcting the heart rate time with time at which a cardiac potential becomes zero on a straight line passing through two points of sampling data at the heart rate time decided in the deciding and sampling data immediately before the sampling data at the heart rate time as reference time; and
calculating a heart rate of the living body from the heart rate time corrected in the correcting.

14. The heart rate detection method according to claim 13, wherein the two points of the sampling data at the heart rate time decided in the deciding and the sampling data immediately before the sampling data at the heart rate time are points on an RS interval from a beginning of a QRS wave composed of a Q wave, an R wave, and an S wave included in the electrocardiogram to a peak of the S wave, and
the correcting corrects the heart rate time with time at which the cardiac potential becomes zero on the RS interval as the reference time.

15. The heart rate detection method according to claim 14, wherein the calculating the heart rate of the living body calculates an R-R interval that is a time interval between an R wave and a previous R wave of the R wave included in the electrocardiogram of the living body using the reference time obtained in the correcting to calculate the heart rate of the living body from the R-R interval.

16. A heart rate detection program causing a computer to execute:
acquiring sensor data indicating an electrocardiogram of a living body to output a sequence of sampling data based on the sensor data;
calculating, every sampling time, a time difference value of sampling data from the sequence of sampling data that is output in the acquiring;
deciding a heart rate time based on time at which a change in the time difference value calculated in the calculating the time difference value exceeds a threshold that is set;
correcting the heart rate time with time at which a cardiac potential becomes zero on a straight line passing through two points of sampling data at the heart rate time decided in the deciding and sampling data immediately before the sampling data at the heart rate time as reference time; and
calculating a heart rate of the living body from the heart rate time corrected in the correcting.

17. The heart rate detection program according to claim 16, wherein the two points of the sampling data at the heart rate time decided in the deciding and the sampling data immediately before the sampling data at the heart rate time are points on an RS interval from a beginning of a QRS wave composed of a Q wave, an R wave, and an S wave included in the electrocardiogram to a peak of the S wave, and
the correcting corrects the heart rate time with time at which the cardiac potential becomes zero on the RS interval as the reference time.

18. The heart rate detection program according to claim 17, wherein the calculating the heart rate of the living body calculates an R-R interval that is a time interval between an R wave and a previous R wave of the R wave included in the electrocardiogram of the living body using the reference time obtained in the correcting to calculate the heart rate of the living body from the R-R interval.
